(19) United States
(12) Patent Application Publication
(10) Pub. No.: US 2025/0033053 A1
(43) Pub. Date: Jan. 30, 2025

(54) CHANNEL CHIP

(71) Applicant: SCREEN Holdings Co., Ltd., Kyoto (JP)

(72) Inventor: Katsuaki KAWARA, Kyoto (JP)

(21) Appl. No.: 18/781,251

(22) Filed: Jul. 23, 2024

(30) Foreign Application Priority Data
Jul. 27, 2023 (JP) 2023-122545

(51) Int. Cl. B01L 3/00

(52) U.S. Cl. CPC B01L 3/502761; B01L 3/50273; B01L 2300/0645; B01L 2300/0816; B01L 2300/0864

(57) ABSTRACT

A channel chip includes a main channel and a plurality of auxiliary channels. A liquid containing particles flows through the main channel. The plurality of auxiliary channels branch from the main channel. A channel width in each of the plurality of auxiliary channels is fixed. Lengths of the plurality of auxiliary channels become shorter toward the downstream side in a flow direction of the liquid flowing through the main channel.

### CROSS REFERENCE TO RELATED APPLICATIONS

The present application claims the benefit of priority to Japanese Patent Application No. 2023-122545 filed on Jul. 27, 2023, and all the description contents described in the Japanese patent application are incorporated herein by reference.

### BACKGROUND

The present disclosure relates to a channel chip.

Conventionally, a channel chip that separates specific cells from blood is known. For example, a channel chip including a main channel and a plurality of branch channels that branch from the main channel is known. Blood serving as a sample liquid is made to flow through the main channel. Particles larger than a predetermined size such as cancer cells present in blood reach the downstream end of the main channel. Blood components other than the particles larger than the predetermined size such as cancer cells flow to the plurality of branch channels. In such a way, the cancer cells, etc. are separated from blood.

In the channel chip described above, the individual branch channel is set to have a first width on the starting end side (main channel side) and set to have a second width larger than the first width on the terminal end side. Also, the closer to the downstream end of the main channel the branch channel is, the closer to the starting end side a position where the first width is switched to the second width is set for each branch channel.

### SUMMARY

In the channel chip as described above, there is a need to appropriately set a channel resistance value for each of the branch channels. Therefore, at the time of designing the channels of the channel chip, a channel resistance value to be set for each branch channel is calculated, and a width and a length, etc. of the branch channel is set based on the results of calculation.

However, in the channel chip described above, each branch channel has a portion having the first width (hereinafter, sometimes referred to as the base end side portion) and a portion having the second width larger than the first width (hereinafter, sometimes referred to as the leading end side portion). Since the channel resistance value of the branch channel is calculated by using a length and a width of the base end side portion and a length and a width of the leading end side portion, design of the channels of the channel chip is complicated.

A channel chip according to an aspect of the present disclosure includes a main channel and a plurality of auxiliary channels. A liquid containing particles flows through the main channel. The plurality of auxiliary channels branch from the main channel. A channel width in each of the plurality of auxiliary channels is fixed. Lengths of the plurality of auxiliary channels become shorter toward a downstream side in a flow direction of the liquid flowing through the main channel.

In a mode of the present disclosure, the channel widths of the plurality of auxiliary channels may be a same size.

In a mode of the present disclosure, a collection portion to collect the liquid having passed through the plurality of auxiliary channels may be provided. The plurality of auxiliary channels may include a most upstream auxiliary channel disposed at a most upstream in the flow direction, and a most downstream auxiliary channel disposed at a most downstream in the flow direction. At least part of the collection portion may be disposed in a rectangular region. Two sides of the rectangular region may be a portion of the main channel between the most upstream auxiliary channel and the most downstream auxiliary channel, and the most upstream auxiliary channel.

In a mode of the present disclosure, a discharge portion may be provided that is connected to the collection portion and that discharges the liquid collected by the collection portion. The entire discharge portion may be disposed in the rectangular region.

In a mode of the present disclosure, a downstream side channel and an electrode may be provided. The downstream side channel may be connected to a downstream end portion of the main channel, and the liquid having passed through the main channel may pass through the downstream side channel. The electrode causes dielectrophoresis of dielectric particles contained in the liquid passing through the downstream side channel.

### BRIEF DESCRIPTION OF THE DRAWINGS

FIG. 1 is a plan view schematically showing a structure of a separation device including an HDF chip according to a preferred embodiment of the present disclosure.

FIG. 2 is a plan view schematically showing a structure around an upstream side joining portion of the HDF chip.

FIG. 3 is a view schematically showing a structure around a plurality of auxiliary channels.

FIG. 4 is a schematic view for explaining the operating principle of an HDF.

FIG. 5 is a schematic view showing a structure around auxiliary channels of a conventional HDF chip.

FIG. 6 is a schematic view showing a channel model having a rectangular parallelepiped shape.

FIG. 7 is a schematic view showing a structure around the auxiliary channels of the HDF chip of the present preferred embodiment.

FIG. 8 is a plan view schematically showing a structure of a separation device including an HDF chip according to a modified example of the present disclosure.

### DETAILED DESCRIPTION

Hereinafter, preferred embodiments of the present disclosure will be described with reference to the drawings. It is noted that in the drawings, the same or corresponding portions will be given the same reference signs and will not be repeatedly described.

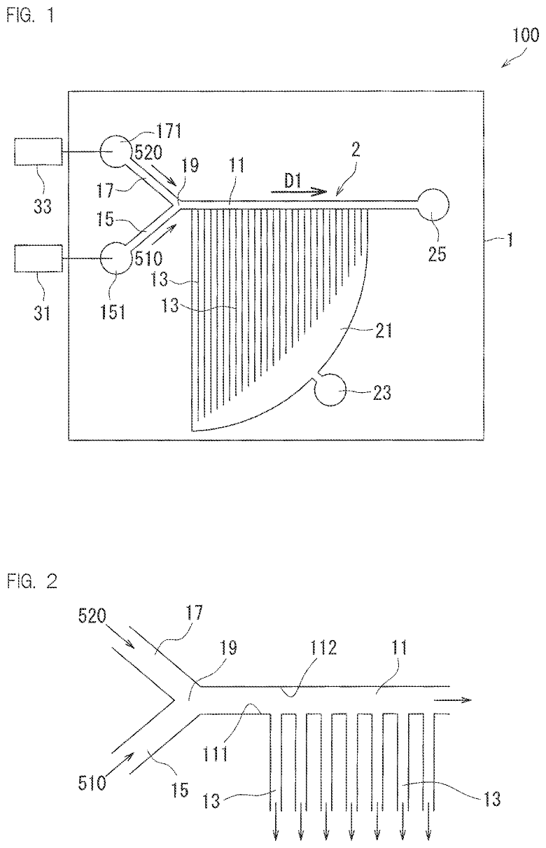

With reference to FIGS. 1 to 7, a separation device 100 including an HDF chip 1 according to a preferred embodiment of the present disclosure will be described. FIG. 1 is a plan view schematically showing a structure of the separation device 100 including the HDF chip 1 according to the preferred embodiment of the present disclosure.

As shown in FIG. 1, a separation device 100 of the present preferred embodiment includes the hydrodynamic filtration (HDF) chip 1. The HDF chip 1 has a function as a hydrodynamic filter. For example, the HDF chip 1 is a micro channel chip that aims at separation and/or concentration of particles. In the present preferred embodiment, the HDF chip 1 can be used for pathological diagnoses, for example. Also, for example, the HDF chip 1 is constituted of a transparent substrate, etc. It is noted that the HDF chip 1 is an example of the “channel chip” of the present disclosure.

The HDF chip 1 includes at least a main channel 11 and a plurality of auxiliary channels 13. In the present preferred embodiment, the HDF chip 1 includes the main channel 11, the plurality of auxiliary channels 13, a sample liquid supply channel 15, a transport liquid supply channel 17, an upstream side joining portion 19, a collection portion 21, a first outlet 23, and a second outlet 25. It is noted that the first outlet 23 is an example of a “discharge portion” of the present disclosure. The main channel 11, the plurality of auxiliary channels 13, the sample liquid supply channel 15, the transport liquid supply channel 17, the upstream side joining portion 19, the collection portion 21, the first outlet 23, and the second outlet 25 are formed by resin layers, etc. disposed on the transparent substrate, for example. Specifically, all or part of a plane in which the main channel 11, the auxiliary channels 13, the sample liquid supply channel 15, the transport liquid supply channel 17, the upstream side joining portion 19, the collection portion 21, the first outlet 23, and the second outlet 25 are arranged is constituted of, for example, a metal, an insulator, a semiconductor, PDMS (dimethyl polysiloxane), glass, and/or a photoresist.

The main channel 11 extends in a straight-line form toward the predetermined direction, for example. A liquid flows through the main channel 11. In the present preferred embodiment, a sample liquid 510 flows through the main channel 11. In the present preferred embodiment, the sample liquid 510 and a transport liquid 520 flow through the main channel 11. The sample liquid 510 is a liquid containing particles. The sample liquid 510 is not particularly limited but is blood, for example. The transport liquid 520 is a liquid not containing particles, for example. The transport liquid 520 is not particularly limited but is a liquid culture media, for example. A grain diameter of the particle is, for example, not more than tens of μm. In the present preferred embodiment, the grain diameter of the particle is, for example, not less than 1 μm and not more than 20 μm.

A shape of the particle is not particularly limited but is preferably a spherical shape, for example. However, the shape of the particle may be a shape other than the spherical shape. Also, in the present preferred embodiment, the particles may be particles made of a single individual piece or may be a group of fine particles made by a plurality of fine particles gathering together to form a single mass. Also, the particles may be formed symmetrically with respect to a point or a line, or may be formed asymmetrically with respect to a point or a line. Also, the particles may not have concavities and convexities on their surfaces, or may have concavities and convexities or their surfaces.

The plurality of auxiliary channels 13 branch from the main channel 11. Specifically, the plurality of auxiliary channels 13 are disposed at substantially equal pitches along the direction in which the main channel 11 extends, for example. The plurality of auxiliary channels 13 extend in a direction intersecting the main channel 11. In the present preferred embodiment, the plurality of auxiliary channels 13 extend in a direction orthogonal to the main channel 11. In the present preferred embodiment, the plurality of auxiliary channels 13 are connected perpendicularly to the main channel 11. However, the plurality of auxiliary channels 13 are preferably connected perpendicularly to the main channel 11, but may be connected at an angle not perpendicular to the main channel 11.

An HDF 2 includes the main channel 11 and the plurality of auxiliary channels 13. Specifically, part of the liquid flowing through the main channel 11 flows into the plurality of auxiliary channels 13. Also, part of the particles contained in the liquid flowing through the main channel 11 flows into the plurality of auxiliary channels 13. Whether or not the particles flow into the plurality of auxiliary channels 13 depends on the grain diameter, for example. However, even if the particles have grain diameters smaller than the channel width of the auxiliary channel 13, the particles will not necessarily flow into the auxiliary channel 13. The particles having the smaller grain diameters more easily flow into the auxiliary channel 13, and the particles having the larger grain diameters less easily flow into the auxiliary channel 13. For example, in a case where blood is used as the sample liquid 510, it is possible to make blood plasma (also called as plasma) serving as a liquid component of blood and red blood cells and blood platelets flow through the auxiliary channel 13, and to inhibit cancer cells and white blood cells contained in blood from flowing through the auxiliary channel 13. That is, it is possible to separate the particles having the large grain diameters such as cancer cells and the white blood cells from blood, for example. The operating principle of the HDF 2 will be described later.

Also, the plurality of auxiliary channels 13 are formed so that channel resistance is gradually reduced from the upstream side of the main channel 11 toward the downstream side. Specifically, in the present preferred embodiment, a channel width in each of the plurality of auxiliary channels 13 is fixed. That is, the channel width in each of the auxiliary channels 13 is fixed from an end portion on the base end side (main channel 11 side) to an end portion on the leading end side (side far from the main channel 11). Therefore, the entire auxiliary channel 13 contributes to separation of the particles. That is, when designing the auxiliary channels 13 as described later, the channel resistance of the entire auxiliary channel 13 is used.

Also, lengths of the plurality of auxiliary channels become shorter toward the downstream side in the flow direction D1 of the liquid flowing through the main channel 11. In other words, the lengths of the auxiliary channels 13 become shorter toward a downstream end of the main channel 11. In the present preferred embodiment, the lengths of the auxiliary channels 13 become gradually shorter toward the downstream end of the main channel 11. It is noted that in the present preferred embodiment, all the auxiliary channels 13 are formed to have different lengths from each other. In other words, all the lengths of the plurality of auxiliary channels 13 are different from each other. Also, in the present preferred embodiment, the fixed channel widths of the plurality of auxiliary channels 13 are the same size. The channel widths of the auxiliary channels 13 are not particularly limited but range approximately from 10 μm to tens of μm, for example. It is noted that in the present preferred embodiment, the type of the particles to be separated is the same in all the auxiliary channels 13.

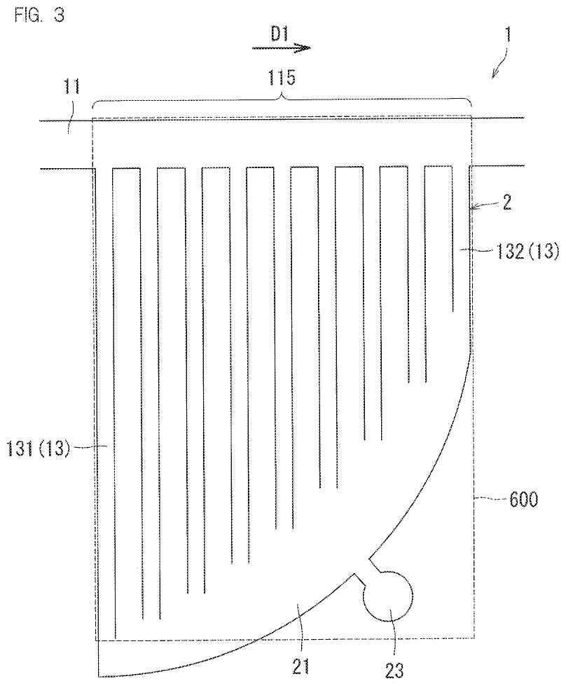

FIG. 2 is a plan view schematically showing a structure around the upstream side joining portion 19 of the HDF chip 1. As shown in FIG. 2, the sample liquid 510 flows through the sample liquid supply channel 15. The sample liquid supply channel 15 is a channel for supplying the sample liquid 510 to the main channel 11. It is noted that although the sample liquid supply channel 15 is inclined with respect to the main channel 11 in the present preferred embodiment, the present disclosure is not limited to this. For example, the sample liquid supply channel 15 may be parallel to the main channel 11 or may be perpendicular to the main channel 11.

The transport liquid 520 flows through the transport liquid supply channel 17. The transport liquid supply channel 17 is a channel for supplying the transport liquid 520 to the main channel 11. The transport liquid 520 is a liquid for transporting the particles larger than a predetermined grain diameter, for example. In other words, the transport liquid 520 is a liquid for transporting the particles not flowing into the auxiliary channels 13 to the downstream side of the main channel 11. It is noted that although the transport liquid supply channel 17 is inclined with respect to the main channel 11 in the present preferred embodiment, the present disclosure is not limited to this. For example, the transport liquid supply channel 17 may be parallel to the main channel 11 or may be perpendicular to the main channel 11.

Also, the sample liquid supply channel 15 is disposed so that the sample liquid 510 flows through a portion of the main channel 11 on the auxiliary channels 13 side. The transport liquid supply channel 17 is disposed so that the transport liquid 520 flows through a portion of the main channel 11 on the opposite side to the auxiliary channels 13. In the present preferred embodiment, the sample liquid supply channel 15 connects to an inner wall 111 on the auxiliary channels 13 side out of inner walls of the main channel 11. The transport liquid supply channel 17 connects to an inner wall 112 on the opposite side to the auxiliary channels 13 out of the inner walls of the main channel 11.

In the upstream side joining portion 19, the sample liquid supply channel 15 and the transport liquid supply channel 17 join together. The upstream side joining portion 19 is disposed at an upstream end of the main channel 11. Therefore, the sample liquid 510 passing through the sample liquid supply channel 15 and the transport liquid 520 passing through the transport liquid supply channel 17 join together in the upstream side joining portion 19 and flows through the main channel 11.

As shown in FIG. 1, the collection portion 21 collects the liquid having passed through the plurality of auxiliary channels 13. The collection portion 21 connects to the plurality of (herein, all of) auxiliary channels 13. Also, the collection portion 21 connects to the first outlet 23. The collection portion 21 collects the liquid having passed through the plurality of auxiliary channels 13, and guides it to the first outlet 23.

The liquid having passed through the plurality of auxiliary channels 13 is recovered in a first recovery portion (not shown) via the collection portion 21 and the first outlet 23. The first recovery portion includes a recovery container, for example.

The second outlet 25 connects to the downstream end of the main channel 11. Therefore, the liquid having passed through the main channel 11 flows to the second outlet 25. At least the particles contained in the sample liquid 510 larger than the predetermined grain diameter flow to the second outlet 25. In the present preferred embodiment, the transport liquid 520 containing the particles larger than the predetermined grain diameter flows. The liquid having passed through the main channel 11 (herein, the transport liquid 520) is recovered in a second recovery portion (not shown) via the second outlet 25. The second recovery portion includes a recovery container, for example.

Next, with reference to FIG. 3, a structure around the plurality of auxiliary channels 13 will be described. FIG. 3 is a view schematically showing the structure around the plurality of auxiliary channels 13. It is noted that in FIG. 3, the number of the auxiliary channels 13 is reduced and illustrated for facilitating understanding.

As shown in FIG. 3, the plurality of auxiliary channels 13 have a most upstream auxiliary channel 131 disposed at the most upstream in the flow direction D1, and a most downstream auxiliary channel 132 disposed at the most downstream in the flow direction D1. The most upstream auxiliary channel 131 is connected to a portion of the main channel 11 at the most upstream in the flow direction D1 in comparison with the other auxiliary channels 13. The most downstream auxiliary channel 132 is connected to a portion of the main channel 11 on the most downstream side in the flow direction D1 in comparison with the other auxiliary channels 13.

Herein, a portion of the main channel 11 disposed between the most upstream auxiliary channel 131 and the most downstream auxiliary channel 132 is provided as a portion 115. A rectangular region whose two sides are the portion 115 and the most upstream auxiliary channel 131 is provided as a region 600. In the present preferred embodiment, at least part of the collection portion 21 is disposed in the region 600. Specifically, a portion of the collection portion 21 excluding a portion around the most upstream auxiliary channel 131 is disposed in the region 600. That is, a large portion of the collection portion 21 is disposed in the region 600.

Also, in the present preferred embodiment, the first outlet 23 is disposed in the region 600. Specifically, the entire first outlet 23 is disposed in the rectangular region 600.

Successively, with reference to FIG. 1, the separation device 100 and the HDF chip 1 will be further described. The separation device 100 further includes a sample liquid sending portion 31 and a transport liquid sending portion 33. The separation device 100 may include a controller that drives the sample liquid sending portion 31 and the transport liquid sending portion 33. The sample liquid sending portion 31 and the transport liquid sending portion 33 are not particularly limited but may be micropumps, for example. The micropumps may be mechanical pumps or may be non-mechanical pumps.

The controller may include a control unit and a storage unit, for example. The control unit has a processor, for example. The control unit may have a central processing unit (CPU) or a general-purpose arithmetic logic unit, for example. The storage unit includes a main storage device and an auxiliary storage device, for example. The main storage device is a semiconductor memory, for example. The auxiliary storage device is a semiconductor memory and/or a hard disk drive, for example. The storage unit may include a removable media.

The sample liquid sending portion 31 connects to the sample liquid supply channel 15, and makes the sample liquid 510 flow through the sample liquid supply channel 15. Specifically, the sample liquid supply channel 15 has a sample liquid inlet 151. The sample liquid inlet 151 is disposed at an upstream end of the sample liquid supply channel 15. The sample liquid sending portion 31 connects to the sample liquid inlet 151. By supplying the sample liquid 510 to the sample liquid inlet 151 by the sample liquid sending portion 31, the sample liquid 510 flows through the sample liquid supply channel 15 and the main channel 11.

The transport liquid sending portion 33 connects to the transport liquid supply channel 17, and makes the transport liquid 520 flow through the transport liquid supply channel 17. Specifically, the transport liquid supply channel 17 has a transport liquid inlet 171. The transport liquid inlet 171 is disposed at an upstream end of the transport liquid supply channel 17. The transport liquid sending portion 33 connects to the transport liquid inlet 171. By supplying the transport liquid 520 to the transport liquid inlet 171 by the transport liquid sending portion 33, the transport liquid 520 flows through the transport liquid supply channel 17 and the main channel 11.

In the present preferred embodiment, the sample liquid sending portion 31 supplies the sample liquid 510 so that the sample liquid 510 becomes a laminar flow. The transport liquid sending portion 33 supplies the transport liquid 520 so that the transport liquid 520 becomes a laminar flow. Therefore, in the present preferred embodiment, each liquid becomes a laminar flow in the HDF chip 1.

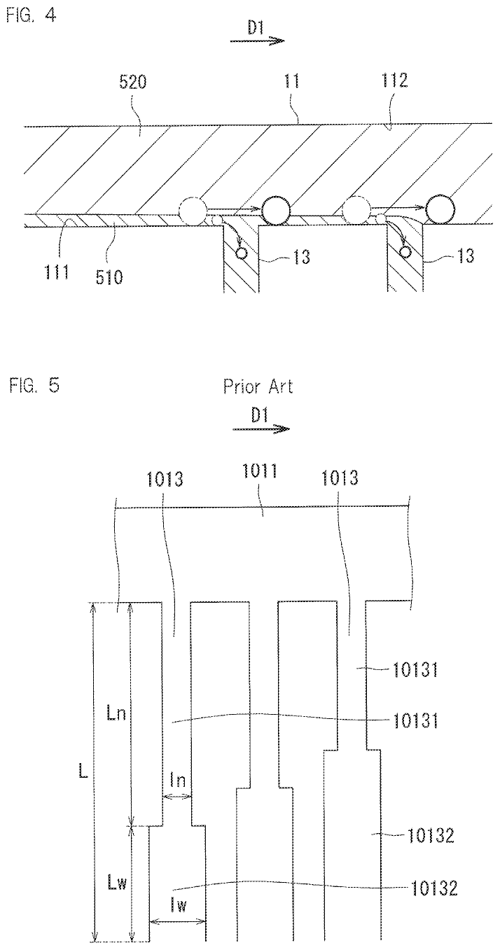

Next, with reference to FIG. 4, the operating principle of the HDF 2 will be described. FIG. 4 is a schematic view for explaining the operating principle of the HDF 2. As shown in FIG. 4, when the sample liquid sending portion 31 supplies the sample liquid 510 and the transport liquid sending portion 33 supplies the transport liquid 520, the sample liquid 510 and the transport liquid 520 flow through the main channel 11. The sample liquid 510 flows along the inner wall 111 on the auxiliary channels 13 side, and the transport liquid 520 flows along the inner wall 112 on the opposite side to the auxiliary channels 13. At this time, centers of various particles pass through positions separated from the inner wall 111 of the main channel 11 by not less than radiuses of the particles.

Herein, a fluid within a predetermined distance from the inner wall 111 of the main channel 11 flows into the auxiliary channels 13. A fluid flowing far from the inner wall 111 of the main channel 11 by more than the predetermined distance, on the other hand, does not flow into the auxiliary channels 13 but goes straight through the main channel 11.

Therefore, the particles having small grain diameters and passing through the vicinity of the inner wall 111 flow into the auxiliary channels 13. The particles having large grain diameters and passing through positions far from the inner wall 111, on the other hand, do not flow into the auxiliary channels 13 but go straight through the main channel 11. That is, under a predetermined condition, the particles larger than a predetermined size are discharged from the main channel 11, whereas the particles not more than the predetermined size are not discharged from the main channel 11. Since discharge from the main channel 11 to the auxiliary channels 13 is repeated by the plurality of auxiliary channels 13, the particles not more than the predetermined size do not reach the downstream end of the main channel 11. It is noted that blood plasma serving as a liquid component does not reach the downstream end of the main channel 11 as well. Therefore, it is possible to separate the particles having the large grain diameters such as cancer cells and the white blood cells from blood, for example, by the HDF chip 1.

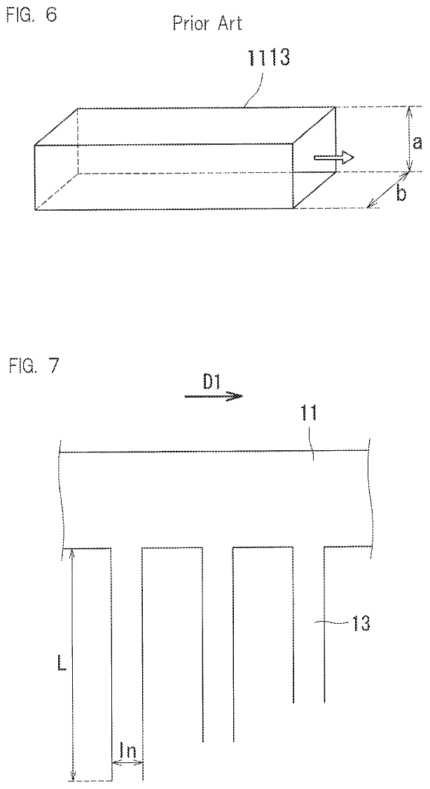

Next, with reference to FIGS. 5 to 7, a method for setting the lengths and channel widths of the auxiliary channels 13 will be described. In other words, a method for designing the auxiliary channels 13 will be described. Herein, in order to facilitate understanding of effects of the present preferred embodiment, a method for designing the auxiliary channels 1013 in a conventional HDF chip will be described first with reference to FIGS. 5 and 6. FIG. 5 is a schematic view showing a structure around the auxiliary channels 1013 of the conventional HDF chip. FIG. 6 is a schematic view showing a channel model having a rectangular parallelepiped shape.

As shown in FIG. 5, in the conventional HDF chip, a portion 10131 of each auxiliary channel 1013 on the base end side (side close to a main channel 1011) is formed to have a first width. A portion 10132 of each auxiliary channel 1013 on the leading end side (side far from the main channel 1011) is formed to have a second width larger than the first width. Toward the downstream side in the flow direction D1, a position where the first width is changed to the second width is disposed on the base end side.

Herein, a length of the auxiliary channel 1013 is provided as L, a length of the portion 10131 is provided as Ln, and a length of the portion 10132 is provided as Lw. Also, a channel width of the portion 10131 is provided as ln, and a channel width of the portion 10132 is provided as lw. Also, the height of the auxiliary channel 1013 is provided as T. It is noted that expressions used in the following description are expressions used in a case where the height T>the channel width ln, and the height T>the channel width lw. However, in a case where the height T≥the channel width ln, for example, in the following expression, f(T, ln) may be f(ln, T). Also, in a case where the height T≥the channel width lw, for example, in the following expression, f(T, lw) may be f(lw, T).

The height T of the auxiliary channel 1013 is a size in the direction perpendicular to the longitudinal direction and the width direction of the auxiliary channel 1013. That is, the height T of the auxiliary channel 1013 is a size in the direction perpendicular to the paper plane of FIG. 5. It is noted that the height T of the auxiliary channel 1013 is fixed. Also, the height of the portion 10131 of the auxiliary channel 1013 and the height of the portion 10132 are each the same height. Also, channel resistance of the auxiliary channel 1013 is provided as R. Also, a proportional constant is provided as k.

In this case, the length Ln of the portion 10131 can be expressed by the following expression (1). Also, the length Lw of the portion 10132 can be expressed by the following expression (2). k>0 and g(T, lw, ln)>0.

$$Ln = k ( R - \frac{L}{f ( T , lw )} ) g ( T , lw , ln ) \qquad ( 1 )$$

$$Lw = k ( \frac{L}{f ( T , ln )} - R ) g ( T , lw , ln ) \qquad ( 2 )$$

It is noted that the function f of the expressions (1) and (2) described above is based on the known expression (3) below. Specifically, as shown in FIG. 6, the larger one of the height and the channel width of the channel 1113 having a rectangular parallelepiped shape is a, and the smaller one of the height and the channel width of the channel 1113 is provided as b. In this case, the function f(a, b) is expressed by the expression (3). It is noted that, for example, the height is provided as a and the channel width is provided as b.

$$f ( a , b ) = {a}^{3} b ( 1 - \frac{192 a}{{π}^{5} b} {\sum }_{n = 1 , 3 , 5 , ⋯}^{∞} \frac{tanh ( n π b / 2 a )}{{n}^{5}} ) \qquad ( 3 )$$

Also, in the expression (1), since the length Ln is a positive value, there is a need for satisfying the following expression (4).

$$R > \frac{L}{f ( T , lw )} \qquad ( 4 )$$

Also, in the expression (2), since the length Lw is a positive value, there is a need for satisfying the following expression (5).

$$\frac{L}{f ( T , ln )} > R \qquad ( 5 )$$

A channel resistance R to be set for each auxiliary channel 1013 is calculated in advance by a known method. Also, the lengths L, the channel widths ln, the channel widths lw, and the heights T of all the auxiliary channels 1013 are equal to each other. That is, the lengths L of all the auxiliary channels 1013 are equal to each other. Also, the channel widths ln of all the auxiliary channels 1013 are equal to each other. Also, the channel widths lw of all the auxiliary channels 1013 are equal to each other. Also, the heights T of all the auxiliary channels 1013 are equal to each other.

Therefore, when designing the auxiliary channels of the conventional HDF chip, the length Ln and the length Lw of each auxiliary channel 1013 are calculated by using the expressions (1) and (2) so that the lengths L, the channel widths ln, the channel widths lw, and that the heights T of all the auxiliary channels 1013 are equal to each other, and the channel resistance R of each auxiliary channel 1013 becomes a predetermined value. At this time, further, there is a need for setting the lengths L, the channel widths ln, the channel widths lw, and the heights T so that the expressions (4) and (5) are satisfied.

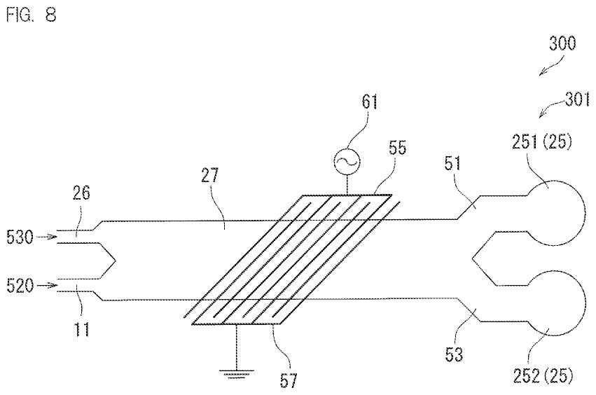

The auxiliary channels 13 of the HDF chip 1 of the present preferred embodiment, on the other hand, can be designed as below. FIG. 7 is a schematic view showing a structure around the auxiliary channels 13 of the HDF chip 1 of the present preferred embodiment.

Specifically, as shown in FIG. 7, the length of the auxiliary channel 13 is provided as L, the channel width is provided as ln, and the height is provided as T. The height T of the auxiliary channel 13 is a size in the direction perpendicular to the longitudinal direction and the width direction of the auxiliary channel 13. That is, the height T of the auxiliary channel 13 is a size in the direction perpendicular to the paper plane of FIG. 7. It is noted that the height T of the auxiliary channel 13 is fixed. The channel width ln of the auxiliary channel 13 is also fixed. Also, channel resistance of the auxiliary channel 13 is provided as R. Also, a proportional constant is provided as k.

In this case, the length L of the auxiliary channel 13 is expressed by the following known expression (6). It is noted that k>0.

$$L = kRf ( T , ln ) \qquad ( 6 )$$

The channel resistance R to be set for each auxiliary channel 13 is calculated in advance by a known method. Also, the lengths L and the heights T of all the auxiliary channels 13 are equal to each other. That is, the lengths L of all the auxiliary channels 13 are equal to each other. Also, the heights T of all the auxiliary channels 13 are equal to each other.

Therefore, when designing the auxiliary channels 13 of the HDF chip 1 of the present preferred embodiment, the length L of each auxiliary channel 13 is calculated by using the expression (6) so that the channel widths ln and the heights T of all the auxiliary channels 13 are equal to each other, and that the channel resistance R of each auxiliary channel 13 becomes a predetermined value. It is noted that in the present preferred embodiment, unlike the case shown in FIG. 5 where the length Ln and the length Lw of the auxiliary channel 1013 are calculated, there is no need for satisfying the restriction conditions shown in the expressions (4) and (5).

In such a way, in the present preferred embodiment, the number of parameters (for example, the length and the channel width) required at the time of designing the auxiliary channels 13 is reduced, and the restriction conditions as shown in the expressions (4) and (5) do not have to be satisfied.

As described with reference to FIGS. 1 to 7 above, in the present preferred embodiment, the channel width in each of the auxiliary channels 13 is fixed. Also, the lengths of the plurality of auxiliary channels 13 become shorter toward the downstream side in the flow direction D1 of the liquid flowing through the main channel 11. Therefore, at the time of designing the channels (of the main channel 11 and the plurality of auxiliary channels 13) of the HDF chip 1, in comparison with the case where the channels of the conventional HDF chip are designed, it is possible to reduce the number of the parameters required for design. Also, at the time of designing the channels of the HDF chip 1, unlike the case where the channels of the conventional HDF chip are designed, the restriction conditions as shown in the expressions (4) and (5) do not have to be satisfied. Therefore, with the HDF chip 1 of the present preferred embodiment, it is possible to prevent channel designing from becoming complicated.

Also, since the restriction conditions as shown in the expressions (4) and (5) do not have to be satisfied, it is possible to improve the degree of freedom in design.

Also, since the lengths of the plurality of auxiliary channels 13 become shorter toward the downstream side in the flow direction D1 of the liquid flowing through the main channel 11, it is possible to decrease an area of the HDF 2. Therefore, it is possible to reduce the amount of material used in the HDF chip 1. Thus, this leads to cost reduction of the HDF chip 1. It is noted that according to trial calculations performed by the applicant of the present application, with the HDF 2 of the HDF chip 1 of the present preferred embodiment, about 43% of the area could be reduced in comparison with the HDF of the conventional HDF chip.

Also, as described above, the channel widths of the plurality of auxiliary channels 13 are the same size. Therefore, it is possible to easily prevent calculation at the time of designing the channels of the HDF chip 1 from becoming complicated.

Also, as described above, at least part of the collection portion 21 is disposed in the rectangular region 600 whose two sides are the portion 115 and the most upstream auxiliary channel 131. Therefore, it is possible to dispose at least part of the collection portion 21 in a region where the auxiliary channels 1013 are disposed in the conventional HDF chip. Thus, it is possible to easily decrease the area of the HDF chip 1. Therefore, it is possible to easily reduce the amount of material used in the HDF chip 1. It is noted that the region 600 corresponds to a region where the HDF (of the main channel 1011 and the plurality of auxiliary channels 1013) is disposed in the conventional HDF chip.

Also, as described above, the entire first outlet 23 is disposed in the rectangular region 600. Therefore, it is possible to further dispose the entire first outlet 23 in the region where the auxiliary channels 1013 are disposed in the conventional HDF chip. Thus, it is possible to further decrease the area of the HDF chip 1. Therefore, it is possible to further reduce the amount of material used in the HDF chip 1.

Next, with reference to FIG. 8, a separation device 300 according to a modified example of the present disclosure will be described. FIG. 8 is a plan view schematically showing a structure of the separation device 300 including an HDF chip 301 according to the modified example of the present disclosure.

As shown in FIG. 8, in the separation device 300 according to the modified example of the present disclosure, the HDF chip 301 includes a main channel 11, a transport liquid supply channel 26, a downstream side channel 27, a first recovery passage 51, and a second recovery passage 53. Also, as well as the HDF chip 1, the HDF chip 301 includes a plurality of auxiliary channels 13, a sample liquid supply channel 15, a transport liquid supply channel 17, an upstream side joining portion 19, a collection portion 21, and a first outlet 23. It is noted that the HDF chip 301 is an example of the “channel chip” of the present disclosure.

A transport liquid 530 flows through the transport liquid supply channel 26. The transport liquid supply channel 26 is a channel for supplying the transport liquid 530 to the downstream side channel 27. The transport liquid 530 is a liquid for transporting particles separated by dielectrophoresis to be described later to the first recovery passage 51, for example. The transport liquid 530 is a liquid not containing particles, for example. The transport liquid 530 is not particularly limited but the same component as the transport liquid 520, for example.

The downstream side channel 27 is connected to a downstream end of the main channel 11 and a downstream end of the transport liquid supply channel 26. For example, a channel width of the downstream side channel 27 is larger than a channel width of the main channel 11 and a channel width of the transport liquid supply channel 26. It is noted that in the modified example, the channel width of the downstream side channel 27 is larger than the sum of the channel width of the main channel 11 and the channel width of the transport liquid supply channel 26. Also, the height of the downstream side channel 27 is the same size as the height of the main channel 11 and the height of the transport liquid supply channel 26.

In such a way, by making the channel width of the downstream side channel 27 larger than the channel width of the main channel 11, it is possible to lower flow speed of the liquid flowing through the downstream side channel 27. Therefore, it is possible to easily separate the particles by the dielectrophoresis to be described later.

The first recovery passage 51 and the second recovery passage 53 are connected to a downstream end of the downstream side channel 27. That is, the downstream side channel 27 is branched into the first recovery passage 51 and the second recovery passage 53.

In the HDF chip 301, a second outlet 25 includes a second outlet 251 and a second outlet 252. The second outlet 251 connects to a downstream end of the first recovery passage 51, and the second outlet 252 connects to a downstream end of the second recovery passage 53.

Herein, in the HDF chip 301, it is possible to further separate the particles flowing through the downstream side channel 27 by the dielectrophoresis (DEP). Specifically, the HDF chip 301 further includes an electrode 55 and an electrode 57 disposed to overlap with the downstream side channel 27. The electrode 55 and the electrode 57 are teeth-shaped electrodes opposing each other. The electrode 55 is connected to an AC source 61, for example. The electrode 57 is grounded, for example. In the modified example, the separation device 300 includes the AC source 61. It is noted that the separation device 300 does not have to include the AC source 61.

Teeth portions of the electrode 55 and the electrode 57 are inclined with respect to the downstream side channel 27 by not less than 10° and not more than 60°, for example. By applying AC voltage between the electrode 55 and the electrode 57 by the AC source 61, it is possible to cause the dielectrophoresis. By adjusting a frequency of the applied AC voltage, it is possible to separate and recover desired particles from plural types of particles passing through the downstream side channel 27.

Specifically, in a case where the plural types of particles passing through the downstream side channel 27 contain dielectric particles, it is possible to separate particular dielectric particles from the plural types of particles by the DEP. For example, cancer cells and white blood cells are dielectric particles. In a case where the plural types of particles passing through the downstream side channel 27 contain the cancer cells and particles larger than a predetermined size, the particles being other than the cancer cells (such as the white blood cells), AC voltage of a particular frequency is applied between the electrode 55 and the electrode 57 so that a dielectrophoresis force more easily acts on the cancer cells, for example. Thereby, since it is possible to move the cancer cells along the electrode 55 and the electrode 57, it is possible to induce only the cancer cells to the second outlet 251 by the dielectrophoresis. Therefore, it is possible to separate the cancer cells and the other particles (such as the white blood cells).

Other structures of the separation device 300, the HDF chip 301, the main channel 11, the plurality of auxiliary channels 13, the sample liquid supply channel 15, the transport liquid supply channel 17, the upstream side joining portion 19, the collection portion 21, and the first outlet 23 according to the modified example of the present disclosure are the same as the separation device 100, the HDF chip 1, the main channel 11, the plurality of auxiliary channels 13, the sample liquid supply channel 15, the transport liquid supply channel 17, the upstream side joining portion 19, the collection portion 21, and the first outlet 23 of the preferred embodiment described above. Also, the other effects of the modified example of the present disclosure are the same as the effects of the preferred embodiment described above.

As described above, the HDF chip 301 according to the modified example of the present disclosure includes the downstream side channel 27 connected to the downstream end of the main channel 11, the downstream side channel through which the liquid having passed through the main channel 11 passes, and the electrode 55 and the electrode 57 that cause the dielectrophoresis of the dielectric particles (herein, the cancer cells) contained in the liquid passing through the downstream side channel 27. Therefore, it is possible to easily separate particular dielectric particles (herein, the cancer cells) from the liquid passing through the main channel 11.

The preferred embodiment of the present disclosure has been described above with reference to the drawings. However, the present disclosure is not limited to the preferred embodiment described above, and can be implemented in various modes within the range not departing from the gist thereof. Also, by appropriately combining the plurality of constituent elements disclosed in the preferred embodiment described above, it is possible to form various disclosures. For example, some constituent elements may be deleted from all the constituent elements shown in the preferred embodiment. Further, the constituent elements of different preferred embodiments may be appropriately combined. The drawings schematically show the respective constituent elements mainly in order to facilitate understanding, and the thickness, the length, the number, the interval, etc. of each constituent element shown in the figures may be different from the actual ones for convenience in creating the drawings. Also, the material, the shape, the size, etc. of each constituent element shown in the preferred embodiment described above are not particularly limited but just an example, and can be variously changed within the range substantially not departing from the effects of the present disclosure.

For example, in the preferred embodiment described above, the example in which blood is used as the sample liquid 510 and the liquid culture media is used as the transport liquid 520 is described. However, the present disclosure is not limited to this. For example, a liquid other than blood may be used as the sample liquid 510 and a liquid other than the liquid culture media may be used as the transport liquid 520.

Also, in the preferred embodiment described above, the particles to be recovered from the second outlet 25 (for example, the cancer cells and the white blood cells) are exemplified as a target to be recovered. However, the present disclosure is not limited to this. For example, particles or a liquid component recovered from the first outlet 23 may be the target to be recovered.

Also, in the modified example described above, the example in which the target particles (for example, the cancer cells) are recovered from the second outlet 251 is described. However, the present disclosure is not limited to this. For example, the target particles may be recovered from the second outlet 252.

Also, in the preferred embodiment described above, the example in which the liquid not containing particles (for example, the transport liquid 520) is used is described. That is, the example in which the liquid not containing particles (for example, the transport liquid 520) flows through the main channel 11 in addition to the liquid containing the particles (for example, the sample liquid 510) is described. However, the present disclosure is not limited to this. For example, only the liquid containing the particles (for example, the sample liquid 510) may flow through the main channel 11. For example, seawater containing particles may be used as the liquid containing the particles (for example, the sample liquid 510) and the particles may be separated from the seawater.

Also, in the preferred embodiment described above, the example in which the liquid containing the particles (for example, the sample liquid 510) and the liquid not containing particles (for example, the transport liquid 520) join together on the HDF chip 1 is described. However, the present disclosure is not limited to this. For example, a mixture liquid in which the liquid containing the particles (for example, the sample liquid 510) and the liquid not containing particles (for example, the transport liquid 520) are mixed may be supplied to the HDF chip 1.

Also, in the preferred embodiment described above, the example in which the liquid passing through all the auxiliary channels 13 is collected in the single collection portion 21 is described. However, the present disclosure is not limited to this. For example, a collection portion that collects a liquid passing through part of auxiliary channels 13 among the plurality of auxiliary channels 13 and a collection portion that collects a liquid passing through the remaining auxiliary channels 13 among the plurality of auxiliary channels 13 may be separately provided. Also, three or more collection portions may be provided. Also, the collection portion and/or the first outlet may be provided.

Also, in the preferred embodiment described above, the example in which each auxiliary channel 13 extends in a straight-line form is described. However, the present disclosure is not limited to this. For example, part or all of the plurality of auxiliary channels 13 may be bent in the middle of the channels.

Also, in the preferred embodiment described above, the example in which the auxiliary channel 13 is connected to one inner wall (inner wall 111) among the inner wall 111 and the inner wall 112 of the main channel 11 is described. However, the present disclosure is not limited to this. For example, the auxiliary channel 13 may be connected to both the inner wall 111 and the inner wall 112.

Also, in the preferred embodiment described above, the example in which a structure such as a sensor is not disposed in the channels (for example, the main channel 11 and the auxiliary channels 13) of the HDF chip 1 is described. However, the present disclosure is not limited to this. For example, a structure such as a physical, electrical, or magnetic sensor may be disposed in the channels (for example, the main channel 11 and the auxiliary channels 13) of the HDF chip 1 as long as it is possible to separate the particles.

Also, in the preferred embodiment described above, the example in which a side wall other than the auxiliary channel 13 is not connected to a side wall in the channel width direction of the auxiliary channel 13 in a leading end of the auxiliary channel 13 as shown in FIG. 7 is described. However, the present disclosure is not limited to this. For example, a side wall expanding from the leading end of the auxiliary channel 13 to the outer side may be formed. However, the side wall connected to the leading end of the auxiliary channel 13 is a side wall that does not effectively contribute to separation of the particles. In other words, a side wall with which channel resistance can be ignored at the time of designing the auxiliary channels 13 may be connected to the leading end of the auxiliary channel 13.

What is claimed is:

1. A channel chip comprising:
a main channel through which a liquid containing particles flows; and
a plurality of auxiliary channels that branch from the main channel, wherein
a channel width in each of the plurality of auxiliary channels is fixed, and lengths of the plurality of auxiliary channels become shorter toward a downstream side in a flow direction of the liquid flowing through the main channel.

2. The channel chip according to claim 1, wherein
the channel widths of the plurality of auxiliary channels are a same size.

3. The channel chip according to claim 1, comprising:
a collection portion to collect the liquid having passed through the plurality of auxiliary channels, wherein
the plurality of auxiliary channels include a most upstream auxiliary channel disposed at a most upstream in the flow direction, and a most downstream auxiliary channel disposed at a most downstream in the flow direction, and
at least part of the collection portion is disposed in a rectangular region whose two sides are a portion of the main channel between the most upstream auxiliary channel and the most downstream auxiliary channel, and the most upstream auxiliary channel.

4. The channel chip according to claim 3, comprising:
a discharge portion, connected to the collection portion, to discharge the liquid collected by the collection portion, wherein
the entire discharge portion is disposed in the rectangular region.

5. The channel chip according to claim 1, comprising:
a downstream side channel, connected to a downstream end portion of the main channel, through which the liquid having passed through the main channel passes; and
an electrode that causes dielectrophoresis of dielectric particles contained in the liquid passing through the downstream side channel.
